(19) United States
(12) Patent Application Publication
(10) Pub. No.: US 2014/0286866 A1
(43) Pub. Date: Sep. 25, 2014

(54) IMAGING AGENTS WITH IMPROVED PHARMACOKINETIC PROFILES

(71) Applicants: ALAN CUTHBERTSON, Oslo (NO); MAGNE SOLBAKKEN, Oslo (NO)

(72) Inventors: ALAN CUTHBERTSON, Oslo (NO); MAGNE SOLBAKKEN, Oslo (NO)

(73) Assignee: GE HEALTHCARE AS, Oslo (NO)

(21) Appl. No.: 14/295,574

(22) Filed: Jun. 4, 2014

Related U.S. Application Data
(63) Continuation of application No. 13/530,498, filed on Jun. 22, 2012, now abandoned.
Application No. 13/530,498 is a continuation of application No. 11/571,313, filed on Dec. 27, 2006.

(51) Int. Cl. A61K 49/00; C07C 317/32

(52) U.S. Cl. CPC A61K 49/0004; C07C 317/32
USPC 424/9.1; 560/13; 564/89

(57) ABSTRACT

The invention relates to compounds suitable for use in an imaging agent said imaging agent showing an improved pharmacokinetic profile.

The invention relates to compounds suitable for use as an imaging agent said imaging agent showing an improved pharmacokinetic profile.

Drug molecules used as therapeutic agents in the treatment of diseases must fulfil certain criteria, e.g. prolonged circulation life time, low immunogenicity and high efficacy. The pharmaceutical industry has for some years now used pegylation, i.e. the modification of a chemical entity by introduction of a polyethylene glycol (PEG) moiety, as a strategy for improving these properties. An extensive overview over pegylation methods and pharmacokinetic/pharmacodynamic results for several PEG-proteins that have been studied in both clinical and research arenas see Advanced Drug Delivery Reviews 55 (2003).

As an example, PEG-Intron™ and Pegasys™ are two products for the treatment of chronic hepatitis which have sales in excess of $ 1 billion per year.

Several examples of pegylated peptides or proteins used for the treatment of diseases exist. The pegylation of a human growth-hormone releasing factor peptide (hGRF) for treatment of growth hormone deficiency has been found to prolong the plasma half-life of the peptide. A single injection of the pegylated peptide produced a sustained pharmacodynamic response.

Yoshioka et al., Biochem. Biophys. Res. Comm 315, 4 (2004) 808-814 describe the optimal site-specific pegylation of mutant TNF-α and show evidence that the molecular shape and weight of the PEG can strongly influence the in vivo antitumour activity of the compound. In this case and almost all other examples of pegylated therapeutic agents, PEG molecules of more than 10 kDa are required to improve efficacy.

In the case of pegylated recombinant staphylokinase however, a new and potentially cost-effective thrombolytic agent for treatment of acute myocardial infarction, a relatively low molecular weight PEG of about 5000 Da has been used. In this case the higher molecular weight PEGs gave too great circulation times. Another example is the pegylation of synthetic GRF1-29 (Sermorelin™) with a PEG of 5000 Da in order to achieve maximum biological activity in vivo (see Advanced Drug Delivery Reviews 55 (2003), 1279-1291). However, these examples of low molecular weight pegylation do appear to be the exceptions to the rule.

Pegylation of peptides and proteins has proven useful in designing new therapeutic agents largely as it improves the poor biopharmaceutical properties of this class of therapeutic agents. In particular, peptides and proteins are known to undergo rapid clearance form the body through proteolysis, renal filtration and liver clearance. Pegylation is considered to be an easy way to prolong the residence time of peptide and protein based therapeutic agents in the bloodstream.

In contrary to peptides and proteins, the clearance of non-peptidic therapeutic agents is a slower process and immunogenicity is often not an issue. This makes pegylation for non-peptidic therapeutic agents less important.

Agents suitable for application in molecular imaging which target specific receptor systems offer great promise in providing important diagnostic and prognostic information to clinicians. Based on an imaging examination the selection of and response to therapy can be controlled with the patient receiving a more personalized approach. This can be achieved by using an imaging agent comprising a vector moiety which has a high affinity to a specific receptor system and an imageable moiety that can be detected in the imaging examination.

The vector moiety of an imaging agent as described in the preceding paragraph may be a therapeutic agent known to target a specific receptor system, e.g. a peptide or pegylated peptide.

In WO-A-01/77145 and WO-A-03/006491 imaging agents are disclosed that comprise a vector moiety to target receptors associated with integrin receptors. These vector moieties are peptide-based compounds which may contain a short PEG moiety to modify pharmacokinetics or blood clearance rates. Due to proteolysis and immunogenicity issues, the use of non-peptidic compounds, e.g. small organic molecules, for therapeutic and diagnostic purposes is generally preferred.

However, the use of small organic molecules known used as therapeutic agents as vector moieties in imaging agents has been met in many circumstances with limited success. This is largely due to the fact that therapeutic agents are designed to possess oral bioavailability and long circulation times in blood to negate the need for multiple daily dosing. These characteristics are quite the opposite of the requirement for imaging agents where rapid excretion from the body is desirable. In addition, as the patient has to be present at the site where the imaging examination takes place (e.g. the site of the scanner), imaging agents can be administered parenterally and the need for bioavailability is removed.

Direct conversion of a therapeutic agent to an imaging agent thus often results in long circulation times and poor biodistribution of the imaging agent. Due to their inherent hydrophobicity, therapeutic agents are typically excreted via the hepatobiliary route rather than the preferred route through the kidneys.

In WO 2004/062568, imaging agents are disclosed that comprise a non-peptidic vector moiety to target Angiotensin II receptors. Several possible linkers might be used to link the non-peptidic vector moiety to an imageable moiety, such as simple bonds, glutaric acid, diglycolic acid or PEG units.

It has now been found that non-peptidic small molecules, e.g. non-peptidic therapeutic agents known to target a specific receptor system, can effectively be used as vector moieties in an imaging agent by introducing a low molecular weight PEG containing moiety into the imaging agent. The resulting imaging agents have improved pharmacokinetic profiles, i.e. the imaging agents are preferably excreted via the renal system.

Thus, viewed from one aspect, the invention provides a compound suitable for use as an imaging agent, said compound consists of
i) a PEG containing moiety having a molecular weight of less than 3000 Da and comprising 2 to 50 ethylene glycol units;
ii) an imageable moiety; and
iii) a non-peptidic vector moiety
   with the proviso that the non-peptidic vector moiety (iii) is not a non-peptidic vector moiety having affinity for the Angiotensin II receptor.

In the context of the present application, a PEG unit is a unit comprising at least two ethylene glycol units. The PEG containing moiety of the compound according to the invention may be a straight chain PEG containing moiety comprising one or more PEG units, the units may or may not be interrupted by a spacer group or functional group or a dendrimeric PEG containing moiety comprising more than one PEG unit.

In a preferred embodiment, the PEG containing moiety of the compound according to the invention is a straight chain PEG containing moiety comprising two or more ethylene glycol units.

The PEG containing moiety of the compound according to the invention has a molecular weight of less than 3000 Da, preferably a molecular weight of less than 2000 Da, more preferably a molecular weight of from 600 to 1000 Da and most preferably a molecular weight of from 120 to 360 Da. It comprises 2 to 50 ethylene glycol units, preferably, 10 to 30 ethylene glycol units and particularly preferably 2 to 6 ethylene glycol units.

In a preferred embodiment, the PEG containing moiety forms a linker between the imageable moiety (ii) and the non-peptidic vector moiety (iii). If serving as a linker, the PEG containing moiety preferably comprises two identical or different functional groups which allow the covalent binding of the PEG containing moiety to the imageable moiety and the non-peptidic vector moiety. Suitable functional groups are for instance amino, hydroxyl, sulfhydryl, carboxyl and carbonyl groups, carbohydrate groups, phenolic and active halogen containing groups. In a preferred embodiment, the PEG containing moiety comprises two different functional groups (heterobifunctional PEG containing moieties).

In another preferred embodiment, the PEG containing moiety is covalently linked to either the imageable moiety (ii) or the non-peptidic vector moiety (iii). In this case, the PEG containing moiety comprises a functional group which allows the covalent binding of the PEG containing moiety to the imageable moiety or the non-peptidic vector moiety. Suitable functional groups are those mentioned in the preceding paragraph.

Preferably, the PEG containing moiety is linked to the imageable moiety (ii) and/or the non-peptidic vector moiety (iii) in such a way that neither the binding affinity of the non-peptidic vector moiety to its target nor the detection of the imageable moiety in the imaging examination is affected by this linkage.

PEG containing moieties that comprise one or more functional groups which may be used for the synthesis of the compounds according to the invention are known in the art and are commercially available. Alternatively, PEG containing moieties can be synthesised by methods known in the art. Briefly, PEG containing moieties can be synthesised from PEG, which may be produced by based catalysed polymerisation of ethylene oxide, giving a distribution of chain lengths and end group modifications depending on the conditions chosen. From the product mixture, low molecular components, like tetraethylene glycol, can be purified by fractional distillation giving homogeneous products. The PEG end groups can be subjected to chemical modifications introducing functional groups like amino, mercaptol, halo, carboxyl and the like suitable for conjugation to other molecules by well known synthetic methods (see for instance S. Zalipsky, Adv. Drug Del. Rev. 16 (1995), 157 and references cited therein).

A preferred PEG containing moiety comprising two functional groups is 17-(Fmoc-amino)-5-oxo-6-aza-3, 9, 12, 15-tetraoxaheptadecanoic acid, the Fmoc-group serving as a protecting group. This PEG containing moiety is especially useful to serve as a linker between an imageable moiety and a non-peptidic vector moiety. Its synthesis is disclosed in detail in Preparation A.

The imageable moiety (ii) of the compounds according to the invention may be any moiety capable of detection either directly or indirectly in an in vivo diagnostic imaging procedure.

The nature of the imageable moiety will depend of the imaging modality utilised in the imaging procedure. The imageable moiety must be capable of detection either directly or indirectly in an in vivo diagnostic imaging procedure, e.g. it must be an imageable moiety which emits or may be caused to emit detectable radiation, e.g. by radioactive decay, fluorescence excitation or spin resonance excitation, an imageable moiety which affects local electromagnetic fields for instance a paramagnetic species or an imageable moiety which absorbs or scatters radiation energy like chromophores. A wide range of suitable imageable moieties are known from e.g. WO-A-98/18496, the content of which is incorporated by reference.

In a preferred embodiment, the imageable moiety (ii) of the compounds according to the invention is an imageable moiety selected from the group consisting of imageable moieties useful in radio imaging, imageable moieties useful in SPECT imaging, imageable moieties useful in PET imaging, imageable moieties useful in MR imaging and imageable moieties useful in optical imaging. In a more preferred embodiment, the imageable moiety (ii) of the compounds according to the invention comprises a radionuclide, a paramagnetic metal ion or a chromophore.

In a first embodiment, the compounds according to the invention comprise an imageable moiety useful in radioimaging and SPECT imaging. Preferably the imageable moiety comprises a gamma emitter with low or no alpha- and beta-emission and with a half-life of more than one hour. In a preferred embodiment, the imageable moiety comprises a radionuclide selected from ⁶⁷Ga, ¹¹¹In, ¹²³I, ¹²⁵I, ¹³¹I, ^{81m}Kr, ⁹⁹Mo, ^{99m}Tc and ²⁰¹Tl. Most preferred is ^{99m}Tc. In a further preferred embodiment, the imageable moiety comprises the aforementioned radionuclides in the form of a chelate complex consisting of the radionuclide and a chelating agent. Such chelating agents are well known from the state of art and typical examples of such chelating agents are described in Table I of WO-A-01/77145.

In a second embodiment, the compounds according to the invention comprise an imageable moiety useful in PET imaging. Preferably the imageable moiety comprises a radioemitter with positron-emitting properties. In a preferred embodiment, the imageable moiety comprises a radionuclide selected from ¹¹C, ¹⁸F, ⁶⁸Ga and ⁸²Rb. ¹⁸F is specifically preferred. When the imageable moiety comprises a metallic radionuclide then these metallic radionuclides are preferably present in the form of chelate complex consisting of the metallic radionuclide and a chelating agent. Such chelating agents are well known from the state of art and typical examples of such chelating agents are described in Table I of WO-A-01/77145. A preferred imageable moiety useful in PET imaging comprises a chelate complex of the chelating agent DOTA and the metallic radionuclide ⁶⁸Ga.

In a third embodiment the compounds according to the invention comprise an imageable moiety useful in MR imaging. Preferably the imageable moiety comprises a paramagnetic metal like those mentioned in U.S. Pat. No. 4,647,447. Preferred paramagnetic metal ions are Gd³⁺, Dy³⁺, Fe³⁺ and Mn²⁺. The imageable moieties comprise the paramagnetic metal ions preferably in the form of a chelate complex consisting of the paramagnetic metal ion and a chelating agent; in particular a chelating agent such as acyclic or cyclic polyaminocarboxylates (e.g. DTPA, DTPA-BMA, DOTA and DO3A) as for instance described in U.S. Pat. No. 4,647,447 and WO-A-86/02841.

In a fourth embodiment the compounds according to the invention comprise an imageable moiety useful in optical imaging. Preferably the imageable moiety comprises a chromophore, i.e. an organic or inorganic group which absorbs and/or emits light. By light is meant electromagnetic radiation having wavelengths from 300-1300 nm. Chromophores having absorption and/or emission maxima in the visible to far infrared range are particularly preferred.

The non-peptidic vector moiety (iii) of the compound according to the invention may be any non-peptidic vector moiety which is capable of target a specific receptor system, with the proviso that the non-peptidic vector moiety is not a non-peptidic vector moiety having affinity for the Angiotensin II receptor.

Suitable receptor systems for targeting with the compounds according to the invention include enzymes such as cyclo-oxygenase, xanthine oxygenase, angiotensin-converting enzyme, dihydrofolate reductase, matrix metalloproteinases, ADP receptors, thrombin receptors, uPA, preferably disease associated receptors where the target is over-expressed on the surface of cells such as angiogenesis-related targets including the integrin family of proteins, uPAR, scavenger receptor on macrophages, growth factor receptors such as VEGF, EGF and PDGF. Other surface receptors of interest include E and P Cadherin and the Selectin family. Representative and non-limiting examples of non-peptidic vector moieties in accordance with the invention include antineoplastic agents such as vincristine, vinblastine, vindesine, busulfan, chlorambucil, spiroplatin, cisplatin, carboplatin, methotrexate, adriamycin, mitomycin, bleomycin, cytosine arabinoside, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (L-asparaginase), etoposide, nystatin, griseofulvin, flucytosine, miconazole, beclomethasone dipropionate, betamethasone, cortisone acetate, dexamethasone, flunisolide, hydrocortisone, methylprednisolone, paramethasone acetate, prednisolone, prednisone, triamcinolone or fludrocortisone acetate; vitamins such as cyanocobalamin or retinoids; antiallergic agents such as amelexanox; non-peptidic inhibitors of tissue factor, non-peptidic compounds downregulating tissue factor expression; non-peptidic inhibitors of platelets such as, GPIa, GPIb and GPIIb-IIIa, von Willebrand factor, prostaglandins, aspirin, ticlopidin, clopigogrel and reopro; non-peptidic inhibitors of coagulation protein targets such as: FIIa FVa, FVIIa, FVIIIA, FIXa, tissue factor, hepatins, hirudin, hirulog, argatroban, DEGR-rFVIIa and annexin V; non-peptidic inhibitors of fibrin formation and promoters of fibrionolysis; non-peptidic antiangiogenic factors such as medroxyprogesteron, pentosan polysulphate, suramin, taxol, thalidomide, non-peptidic metalloproteinase inhibitors; circulatory drugs such as propranolol; metabolic potentiators such as glutathione; antituberculars such as p-aminosalicylic acid, isoniazid, capreomycin sulfate, cyclosexine, ethambutol, ethionamide, pyrazinamide, rifampin or streptomycin sulphate; antivirals such as acyclovir, amantadine, azidothymidine, ribavirin or vidarabine; blood vessel dilating agents such as diltiazem, nifedipine, verapamil, erythritol tetranitrate, isosorbide dinitrate, nitroglycerin or pentaerythritol tetranitrate; antibiotics such as dapsone, chloramphenicol, neomycin, cefaclor, cefadroxil, cephalexin, cephradine, erythromycin, clindamycin, lincomycin, amoxicillin, ampicillin, bacampicillin, carbenicillin, dicloxacillin, cyclacillin, picloxacillin, hetacillin, methicillin, nafcillin, penicillin, polymyxin or tetracycline; antiinflammatories such as diflunisal, ibuprofen, indomethacin, meclefenamate, mefenamic acid, naproxen, phenylbutazone, piroxicam, tolmetin, aspirin or salicylates; antiprotozoans such as chloroquine, metronidazole, quinine or meglumine antimonate; antirheumatics such as penicillamine; narcotics such as paregoric; opiates such as codeine, morphine or opium; cardiac glycosides such as deslaneside, digitoxin, digoxin, digitalin or digitalis; neuromuscular blockers such as atracurium mesylate, gallamine triethiodide, hexafluorenium bromide, metocurine iodide, pancuronium bromide, succinylcholine chloride, tubocurarine chloride or vecuronium bromide; sedatives such as amobarbital, amobarbital sodium, apropbarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, secobarbital sodium, talbutal, temazepam or triazolam; local anaesthetics such as bupivacaine, chloroprocaine, etidocaine, lidocaine, mepivacaine, procaine or tetracaine; general anaesthetics such as droperidol, etomidate, fentanyl citrate with droperidol, ketamine hydrochloride, methohexital sodium or thiopental and pharmaceutically acceptable salts (e.g. acid addition salts such as the hydrochloride or hydrobromide or base salts such as sodium, calcium or magnesium salts) or derivatives (e.g. acetates) thereof. Other examples of non-peptidic vector moieties are nucleic acids, RNA, and DNA of natural or synthetic origin, including recombinant RNA and DNA.

Preferred are non-peptidic inhibitors of tissue factor, non-peptidic compounds downregulating tissue factor expression; non-peptidic inhibitors of platelets non-peptidic inhibitors of fibrin formation and promoters of fibrionolysis

In a preferred embodiment, the non-peptidic vector moiety of the compounds according to the invention is a small organic molecule, preferably a small organic molecule with a molecular weight of less than 1000 Da.

From a further aspect, the invention provides a compound consisting of
i) a PEG containing moiety having a molecular weight of less than 3000 Da and comprising 2 to 50 ethylene glycol units;
ii) an imageable moiety; and
iii) a non-peptidic vector moiety
   with the proviso that the non-peptidic vector moiety (iii) is not a non-peptidic vector moiety having affinity for the Angiotensin II receptor for use in an imaging agent.

Another aspect of the invention is an imaging agent comprising a compound consisting of
i) a PEG containing moiety having a molecular weight of less than 3000 Da and comprising 2 to 50 ethylene glycol units;
ii) an imageable moiety; and
iii) a non-peptidic vector moiety
   with the proviso that the non-peptidic vector moiety (iii) is not a non-peptidic vector moiety having affinity for the Angiotensin II receptor and one or more pharmaceutically acceptable adjuvants, excipients or diluents.

Suitable pharmaceutically acceptable diluents are for instance water, aqueous salt solutions like saline or buffers.

Suitable adjuvants are for instance solubilizers like cyclodextrins, surfactants like Pluronic or Tween, stabilizers or antioxidants like ascorbic acid, gentisic acid or p-aminobenzoic acid or bulking agents for lyophilisation like sodium chloride or mannitol.

Yet another aspect of the invention is an imaging agent comprising a compound consisting of
i) a PEG containing moiety having a molecular weight of less than 3000 Da and comprising 2 to 50 ethylene glycol units;
ii) an imageable moiety; and
iii) a non-peptidic vector moiety
   with the proviso that the non-peptidic vector moiety (iii) is not a non-peptidic vector moiety having affinity for the Angiotensin II receptor, and one or more pharmaceutically acceptable adjuvants, excipients or diluents for use in enhancing image contrast in in vivo imaging, preferably in in vivo imaging of the human or non-human animal body.

In order to achieve enhancement of image contrast in in vivo imaging, the imaging agents according to the invention must be administered in an effective amount. The effective amount depends on various factors like imaging modality and nature of the imageable moiety. Generally, where the imageable moiety comprises a chelated metal ion, dosages of from 0.001 to 5.0 mmoles of chelated metal ion per kilogram of patient bodyweight are effective to achieve adequate enhancement of image contrast. Where the imageable moiety comprises a radionuclide, dosages of 0.01 to 50 mCi per 70 kg bodyweight will normally be sufficient.

Yet a further aspect of the invention is a method of generating contrast enhanced images of a human or non-human animal body wherein an imaging agent comprising a compound consisting of
i) a PEG containing moiety having a molecular weight of less than 3000 Da and comprising 2 to 50 ethylene glycol units;
ii) an imageable moiety; and
iii) a non-peptidic vector moiety
   with the proviso that the non-peptidic vector moiety (iii) is not a non-peptidic vector moiety having affinity for the Angiotensin II receptor, and one or more pharmaceutically acceptable adjuvants, excipients or diluents is used to achieve said contrast enhancement.

The PEG containing moiety, the imageable moiety and the vector moiety can be conjugated using all the known methods of chemical synthesis. Particularly useful is the nucleophile substitution reaction where a leaving group on either moiety is replaced by a nucleophilic group on one of the other moieties. Such a leaving group may be a bromide attached in alpha position to a carbonyl group, and such a nucleophile may be nitrogen.

The imageable moiety and the vector moiety can be conjugated directly to each other with a further conjugation of the PEG containing moiety to either of them using the methods as described above.

The abbreviations used below have the following meanings:

F-moc—9-fluorenylmethoxycarbonyl

THF—tetrahydrofuran

TFA—trifluoroacetic acid

DMF—dimethylformamide

HATU—O-Benzotriazol-1-yl-N,N,N′,N′-tetramethyluronium hexafluorophosohate

DIEA—diisopropylethylamine

DMSO—dimethyl sylphoxide

### Preparation A

### 17-(Fmoc-amino)-5-oxo-6-aza-3,9,12,15-tetraoxaheptadecanoic acid

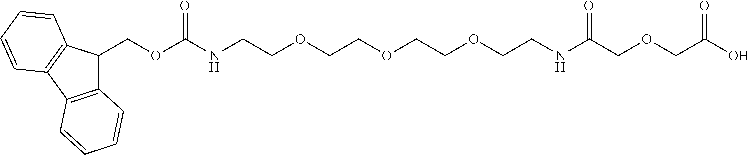 [SMILES: O=C(O)COCC(=O)NCCOCCOCCOCCNC(=O)OCC1c2ccccc2-c2ccccc21]

### A.1 11-Diazido-3,6,9-trioxaundecane

A solution of dry tetraethylene glycol (19.4 g, 0.100 mol) and methanesulphonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stiffing was continued for 4 hrs. Water (60 ml) was added. To the mixture was added sodium hydrogencarbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

### A.2 11-Azido-3,6,9-trioxaundecanamine

To a mechanically, vigorously stirred suspension of 1,11-diazido-3,6,9-trioxaundecane of Preparation A.1 (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0 g (88%) of yellow oil. Analysis by MALDI-TOF mass spectroscopy (matrix: α-cyano-4-hydroxycinnamic acid) gave a M+H peak at 219 as expected. Further characterisation using ¹H (500 MHz) and ¹³C (125 MHz) NMR spectroscopy verified the structure.

### A.3 17-Azido-5-oxo-6-aza-3,9,12,15-tetraoxaheptadecanoic acid

To a solution of 11-azido-3,6,9-trioxaundecanamine (10.9 g, 50.0 mmol) of Preparation A.2 in dichloromethane (100 ml) was added diglycolic anhydride (6.38 g, 55.0 mmol). The reaction mixture was stirred overnight. HPLC analysis (column Vydac 218TP54; solvents: A=water/0.1% TFA and B=acetonitrile/0.1% TFA; gradient 4-16% B over 20 min; flow 1.0 ml/min; UV detection at 214 and 284 nm), showed complete conversion of starting material to a product with retention time 18.3 min. The solution was concentrated to give quantitative yield of a yellow syrup. The product was analysed by LC-MS (ES ionisation) giving [MH]+ at 335 as expected. ¹H (500 MHz) and ¹³C (125 MHz) NMR spectroscopy was in agreement with structure. The product was used in the next step without further purification.

### A.4 17-Amino-5-oxo-6-aza-3,9,12,15-tetraoxaheptadecanoic acid

A solution of 17-azido-5-oxo-6-aza-3,9,12,15-tetraoxaheptadecanoic acid of Preparation A.3 (8.36 g, 25.0 mmol) in water (100 ml) was reduced using H₂(g)-Pd/C (10%). The reaction was run until LC-MS analysis showed complete conversion of starting material (column Vydac 218TP54; solvents: A=water/0.1% TFA and B=acetonitrile/0.1% TFA; gradient 4-16% B over 20 min; flow 1.0 ml/min; UV detection at 214 and 284 nm, ES ionisation giving M+H at 335 for starting material and 309 for the product). The solution was filtered and used directly in the next step.

### A.5 17-(Fmoc-amino)-5-oxo-6-aza-3,9,12,15-tetraoxaheptadecanoic acid

To the aqueous solution of 17-amino-5-oxo-6-aza-3,9,12,15-tetraoxaheptadecanoic acid of Preparation A.4 (corresponding to 25.0 mmol amino acid) was added sodium bicarbonate (5.04 g, 60.0 mmol) and dioxan (40 ml). A solution of Fmoc-chloride (7.11 g, 0.275 mol) in dioxan (40 ml) was added dropwise. The reaction mixture was stirred overnight. Dioxan was evaporated off (rotavapor) and the aqueous phase was extracted with ethyl acetate. The aqueous phase was acidified by addition of hydrochloric acid and precipitated material was extracted into chloroform. The organic phase was dried (MgSO₄), filtered and concentrated to give 11.3 g (85%) of a yellow syrup. The structure was confirmed by LC-MS analysis (column Vydac 218TP54; solvents: A=water/0.1% TFA and B=acetonitrile/0.1% TFA; gradient 40-60% B over 20 min; flow 1.0 ml/min; UV detection at 214 and 254 nm, ES ionisation giving M+H at 531 as expected). The analysis showed very low content of side products and the material was used without further purification.

### Preparation B

### 3-[(4′-Fluorobiphenyl-4-sulfonyl)-(1-hydroxycarbamoylcyclo-pentyl)amino]propionic acid

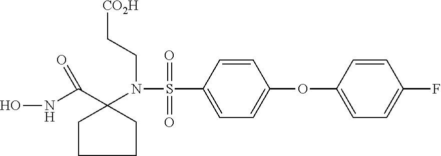 [SMILES: O=C(O)CCN(C1(C(=O)NO)CCCC1)S(=O)(=O)c1ccc(Oc2ccc(F)cc2)cc1]

3-R4′-Fluorobiphenyl-4-sulfonyl)-(1-hydroxycarbamoylcyclopentyl)-aminol-propionic acid (hereinafter referred to as compound 1) was synthesized according to the following multi-step synthesis:

### B.1 Step A

To a solution of 1-aminocyclopentanecarboxylic acid benzyl ester p-toluenesulfonic acid salt (12.1 grams, 30.9 mmol) and triethylamine (10.0 mL, 72 mmol) in water (150 mL) and 1,4-dioxane (150 mL) was added 4′-fluorobiphenyl-4-sulfonyl chloride (8.8 grams, 32.5 mmol). The mixture was stirred at room temperature for 16 hours and then most of the solvent was removed by evaporation under vacuum. The mixture was diluted with ethyl acetate and was washed successively with dilute hydrochloric acid solution, water, and brine. The solution was dried over magnesium sulfate and concentrated to leave 1-(4′-fluorobiphenyl-4-sulfonylamino)cyclopentanecarboxylic acid benzyl ester as a solid, 12.33 grams (76%).

### B.2 Step B

To a solution of 1-(4′-fluorobiphenyl-4-sulfonylamino)cyclopentanecarboxylic acid benzyl ester (23.0 grams, 50.7 mmol) in dry DMF (500 ml) at room temperature was added potassium hexamethyldisilazide (12.2 grams, 61.1 mmole) and, after 45 minutes, tert-butyl-(3-iodopropoxy)dimethylsilane (18.3 grams, 60.9 mmol). The resulting mixture was stirred at room temperature for 16 hours. Additional potassium hexamethyldisilazide (3.0 grams, 15 mmole) and tert-butyl-(3-iodopropoxy)-dimethylsilane (4.5 grams, 15 mmol) were then added. Stirring at room temperature was continued for a further 5 hours. The mixture was quenched by addition of saturated ammonium chloride solution. The DMF was removed by evaporation under vacuum. The residue was taken up in diethyl ether and washed successively with water, dilute aqueous hydrochloric acid solution and brine. After drying over magnesium sulfate, the diethyl ether was evaporated to afford a yellow oil. To this was added hexane and methylene chloride to induce crystallization of the starting material which was recovered by filtration. Evaporation of solvents from the filtrate afforded crude 1-[[3-(tert-butyl-dimethylsilanyloxy)propyl)-(4′-fluorobiphenyl-4-sulfonyl)amino]-cyclopentanecarboxylic acid benzyl ester as an amber oil (27.35 grams).

### B.3 Step C

To a solution of the crude 1-[[3-(tert-butyl-dimethylsilanyloxy)propyl]-(4′-fluorobiphenyl-4-sulfonyl)amino]cyclopentanecarboxylic acid benzyl ester (27.35 grams) in methylene chloride (450 mL) at room temperature was added boron trifluoride etherate (11 mL, 89.4 mmol). After 45 minutes, the reaction was quenched by sequential addition of saturated ammonium chloride solution and water. The organic phase was separated, washed with water and brine and dried over magnesium sulphate. Evaporation of the solvent under vacuum provided crude 1-[(4′-fluorobiphenyl-4-sulfonyl)-(3-hydroxypropyl)amino]-cyclopentane carboxylic acid benzyl ester as an amber oil (22.1 grams).

### B.4 Step D

A solution of the crude 1-[(4′-fluorobiphenyl-4-sulfonyl)-(3-hydroxypropyl)amino]-cyclopentanecarboxylic acid benzyl ester (22.1 grams) in acetone (400 mL) was cooled in an ice bath and treated with Jones reagent (about 20 mL) until an orange colour persisted. The mixture was stirred from 0° C. to room temperature over 2 hours. After quenching excess oxidant with isopropanol (1 mL), Celite® was added and the mixture was filtered. The filtrate was concentrated under vacuum. The residue was taken up in ethyl acetate, washed with water and brine, dried over magnesium sulphate and concentrated to afford crude 1-[(2-carboxyethyl)-(4′-fluorobiphenyl-4-sulfonyl)amino]-cyclopentanecarboxylic acid benzyl ester as an oil (21.4 grams).

### B.5 Step E

To a solution of the crude 1-[(2-carboxyethyl)-(4′-fluorobiphenyl-4-sulfonyl)amino]-cyclopentanecarboxylic acid benzyl ester (21.4 grams) in DMF (500 mL) at room temperature was added potassium carbonate (22.5 grams, 163 mmol) and methyl iodide (3.7 mL, 59.4 mmol). The mixture was stirred for 16 hours at room temperature and was then concentrated under vacuum. The residue was taken up in water and acidified using 6N aqueous hydrogen chloride solution. The resulting mixture was extracted with a mixture of diethyl ether and ethyl acetate. The organic extract was washed with water and brine, dried over magnesium sulphate. After concentration to an amber oil, 1-[(4′-fluorobiphenyl-4-sulfonyl)-(2-methoxycarbonylethyl)amino]-cyclopentane-1-carboxylic acid benzyl ester (12.6 grams), a white solid, was isolated by flash chromatography on silica gel eluting with 15% ethyl acetate in hexane.

### B.6 Step F

A solution of 1-[(4′-fluorobiphenyl-4-sulfonyl)-(2-methoxycarbonylethyl)amino]-cyclopentane-1-carboxylic acid benzyl ester (12.1 grams, 22.4 mmole) in methanol (270 mL) was treated with 10% palladium on activated carbon and hydrogenated in a Parr® shaker at 3 atmospheres pressure for 3.5 hours. After filtration through nylon (pore size 0.45 μm) to remove the catalyst, the solvent was evaporated to afford 1-{(4′-fluorobiphenyl-4-sulfonyl)-(2-methoxycarbonylethyl)amino]cyclopentane-1-carboxylic acid as a white foam (10.1 grams, 100%).

### B.7 Step G

Diisopropylethylamine (4.3 mL, 24.6 mmol) and (benzotriazol-1-yloxy)tris-(dimethylamino)phosphonium hexafluorophosphate (11.0 grams, 24.9 mmol) were added sequentially to a solution of 1-[(4′-fluorobiphenyl-4-sulfonyl)-(2-methoxycarbonylethyl)-amino]cyclopentane-1-carboxylic acid (10.1 grams, 22.4 mmole) in N,N-dimethylformamide (170 mL). The mixture was stirred for 4 hours. Additional diisopropylethylamine (7.8 mL, 44.6 mmol) and O-benzylhydroxylamine hydrochloride (4.64 grams, 29.1 mmol) were then added and the resulting mixture was stirred at 60° C. for 16 hours. After concentration under vacuum, the residue was taken up in water and acidified with 1N aqueous hydrogen chloride solution. The mixture was extracted with ethyl acetate and the extract was washed sequentially with water, saturated aqueous sodium bicarbonate solution and brine. The solution was dried over magnesium sulphate and concentrated to give a solid which upon trituration with 7:3:1 hexane/ethyl acetate/methylene chloride provided 3-[(1-benzyloxycarbamoylcyclopentyl)-(4′-fluorobiphenyl-4-sulfonyl)amino]propionic acid methyl ester as a white crystalline solid (10.65 grams, 86%).

### B.8 Step H

A solution of 3-[(1-benzyloxycarbamoylcyclopentyl)-(4′-fluorobiphenyl-4-sulfonyl)amino]propionic acid methyl ester (10.65 grams, 19.2 mmol) in methanol (250 mL) was treated with 5% palladium on barium sulphate and hydrogenated in a Parr™ shaker at 3 atmospheres pressure for 3 hours. After filtration through nylon (pore size 0.45 μm) to remove the catalyst, the solvent was evaporated to afford 3-[(4′-fluorobiphenyl-4-sulfonyl)-(1-hydroxycarbamoylcyclopentyl)amino)propionic acid methyl ester as a white foam (8.9 grams, 100%). ¹H NMR (DMSO-d₆) δ 8.80 (br s, 1H), 7.85-7.75 (m, 6H), 7.32-7.25 (m, 2H), 3.54 (s, 3H), 3.52-3.48 (m, 2H), 2.73-2.69 (m, 2H), 2.24-2.21 (m, 2H), 1.86-1.83 (m, 2H), 1.60-1.40 (m, 4H).

### B.9 Step I

A solution of 3-[(4′-fluorobiphenyl-4-sulfonyl)-(1-hydroxycarbamoylcyclopentyl)-amino]-propionic add methyl ester (8.9 grams, 19.2 mmol) in methanol (500 mL) was treated with aqueous 1 N sodium hydroxide solution (95 mL, 95 mmol) and stirred at room temperature for 5.5 hours. The mixture was concentrated to remove methanol, diluted with water, acidified with 6 N aqueous hydrochloric acid solution and extracted with ethyl acetate. After washing with water and brine the organic extract was dried over magnesium sulphate and concentrated to afford 3-[(4′-fluorobiphenyl-4-sulfonyl)-(1-hydroxycarbamoyl-cyclopentyl)amino]propionic acid as a white foam which was crystallised from ethyl acetate (6.74 grams, 78%). Mp: 163-164° C.

¹H NMR (DMSO-d₆) δ 12.30 (br s, 1H), 10.40 (br s, 1H), 8.77 (br s, 1H), 7.89-7.74 (m, 6H), 7.31-7.27 (m, 2H), 3.51-3.44 (m, 2H), 2.64-2.60 (m, 2H), 2.24-2.22 (m, 2H), 1.86-1.83 (m, 2H), 1.60-1.40 (m, 4H). MS 449 (M−1).

### Preparation C

### Compound 2

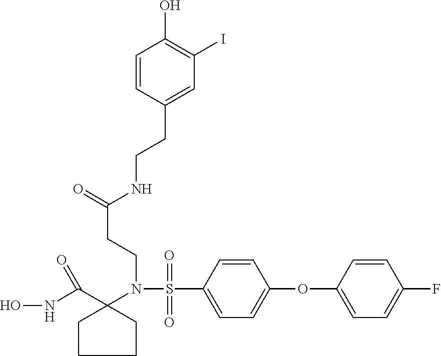 [SMILES: O=C(CCN(C1(C(=O)NO)CCCC1)S(=O)(=O)c1ccc(Oc2ccc(F)cc2)cc1)NCCc1ccc(O)c(I)c1]

### C.1 Step A

2 ml of a 1N iodine solution was added slowly at room temperature to 20 ml of a tyramine solution (50 mM in 30% ammonia). After 5 hours the solution was concentrated to 5 ml and left overnight at 0° C. The off-white precipitate formed was filtrated and washed with cold water. The solid was dried under high vacuum overnight.

¹H NMR (CD₃OD): δ 2.7 (2H, t, J=7 Hz); 2.9 (2H, t, J=7 Hz); 6.7 (1H, d, J=8.1 Hz); 7.1 (1H, dd, J=2.2 Hz, 8 Hz); 7.5 (1H, d, J=2.2 Hz).

### C.2 Step B

To a stirred solution of compound 1, O-(1H-Benzotriazol-1-yl)-N, N, N′, N′-tetramethyluronium tetrafluoroborate (TBTU) and N-methylmorpholine in DMF was added 3-iodotyramine (from Step A). The reaction mixture was allowed to react for 24 hours at room temperature under inert atmosphere. The reaction was monitored via HPLC. After completion the yellow clear solution was concentrated and dried under high vacuum for four hours. The crude product was purified by preparative HPLC and yielded 10% of an off white solid. MS (ESI): 712 (MH⁺) 734 (MNa⁺).

### EXAMPLE 1

### Compound 3

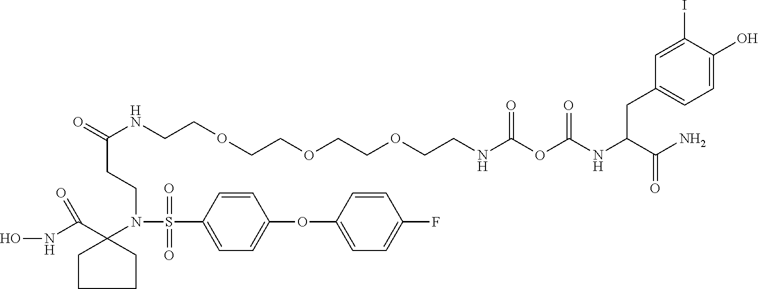 [SMILES: NC(=O)C(Cc1ccc(O)c(I)c1)NC(=O)OC(=O)NCCOCCOCCOCCNC(=O)CCN(C1(C(=O)NO)CCCC1)S(=O)(=O)c1ccc(Oc2ccc(F)cc2)cc1]

Compound 3 was synthesised using a manual nitrogen bubbler apparatus on a 0.05 mmol scale using Fmoc-protected Rink Amide MBHA resin (Novabiochem), Fmoc-3-iodo-Tyr-OH (Novabiochem), Fmoc PEG propionic acid (Polypure AS, Cat 15137-1195) and compound 1. All acid functions were pre-activated prior to amide bond formation using HATU/DIEA as coupling reagents with DMF as solvent. Reaction steps were analysed by Kaiser test. Fmoc-deprotection was carried out using 20% piperidine in DMF treating first for 5 minutes followed by 40 minutes with fresh piperidine solution. The cleavage from the resin was carried out in TFA containing 2.5% H₂O and 2.5% triisopropylsilane for 2 hours. Crude material was purified by preparative HPLC (column Phenomenex Luna C18(2) 10 nm 250×10 mm; solvents A=water/0.1% TFA and B=acetonitrile/0.1% TFA; gradient 20-40% B over 60 min; flow 5.0 ml/min; UV detection at 214 nm) to give 5-10 mg of white solid. Analysis by LC-MS (column Phenomenex Luna C18(2) 3 nm 2.0×50 mm, solvents: A=water/0.1% TFA and B=acetonitrile/0.1% TFA; gradient 10-80% B over 10 min; flow 0.3 ml/min, UV detection at 214 and 254 nm, ESI-MS positive mode) gave peak expected for [MH]⁺.

### EXAMPLE 2

### Compound 4

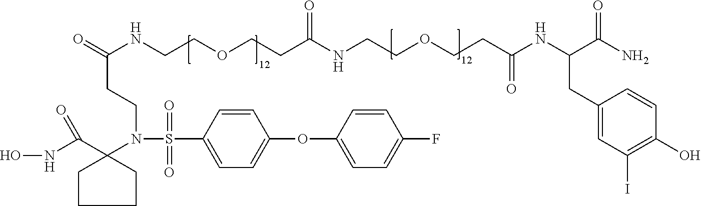

Compound 4 was synthesised using a manual nitrogen bubbler apparatus on a 0.05 mmol scale using Fmoc-protected Rink Amide MBHA resin (Novabiochem), Fmoc-3-iodo-Tyr-OH (Novabiochem), Fmoc-amino-PEG-diglycolic acid (Polypure AS, Cat. 15131-0295) and compound 1. All acid functions were pre-activated prior to amide bond formation using HATU/DIEA as coupling reagents with DMF as solvent. Reaction steps were analysed by Kaiser test. Fmoc-deprotection was carried out using 20% piperidine in DMF treating first for 5 minutes followed by 40 minutes with fresh piperidine solution. The cleavage from the resin was carried out in TFA containing 2.5% H₂O and 2.5% triisopropylsilane for 2 hours. Crude material was purified by preparative HPLC (column Phenomenex Luna C18(2) 10 nm 250×10 mm; solvents A=water/0.1% TFA and B=acetonitrile/0.1% TFA; gradient 20-40% B over 60 min; flow 5.0 ml/min; UV detection at 214 nm) to give 5-10 mg of white solid. Analysis by LC-MS (column Phenomenex Luna C18(2) 3 nm 2.0×50 mm, solvents: A=water/0.1% TFA and B=acetonitrile/0.1% TFA; gradient 10-80% B over 10 min; flow 0.3 ml/min, UV detection at 214 and 254 nm, ESI-MS positive mode) gave peak expected for [MH]⁺.

### EXAMPLE 3

### Radiolabelled Compounds 2, 3 and 4

The identical set of compounds described in Preparation C, Examples 1 and 2 were synthesised where the 3-iodo-tyrosine residue was exchanged for tyrosine. The tyrosine residue was then labelled as the final step using the general procedure described below:

10 μL 0.1 mM Na¹²⁷I (in 0.01M NaOH, 1×10⁻⁹ mol) was added to a vial containing 200 μL 0.2M NH₄OAc buffer (pH 4). This solution was added to the vial containing Na¹²³I (in 0.05M NaOH), which was then transferred to a silanised reaction P15 vial. 5 μL (2.5×10⁻⁸ mol) of a freshly prepared peracetic acid solution in water (approx. 5 mM) was added to the reaction vial. 17 μL (5×10⁻⁸ mol) of a 3 mM solution of substrate in MeOH was added to the reaction vial and the solution mixed by pipetting. The compounds were purified by HPLC, solvent A: 0.1% TFA in water and solvent B: 0.1% TFA in MeCN using a Phenomenex Luna 5 μm C18(2) 150×4.6 mm column. The following gradient was applied:

| Time | % B |
|---|---|
| 0.0 | 30 |
| 20.0 | 70 |
| 20.20 | 100 |
| 23.20 | 100 |
| 23.70 | 30 |
| 30.0 | 30 |

### EXAMPLE 4

Biodistribution of radiolabelled compounds 2, 3 and 4 Radiolabelled compounds 2-4 comprise ¹²⁷I as an imageable moiety and residue X

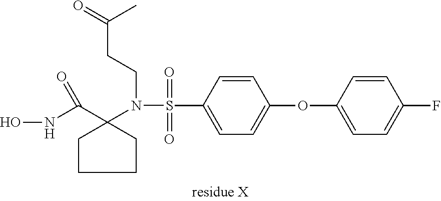 [SMILES: CC(=O)CCN(C1(C(=O)NO)CCCC1)S(=O)(=O)c1ccc(Oc2ccc(F)cc2)cc1]

as a non-peptidic vector moiety that is an inhibitor for matrix metalloproteinase. Compounds 2 and 3 do not comprise PEG moities according to the invention and serve as comparison compounds.

Biodistribution studies were carried out in a Lewis Lung mouse Carcinoma model as described in Bae et al, Drugs Exp Clin Res, 2003; 29 (1): 15-23. Following tail vein injection of the compounds, e the excretion profile was monitored at 5, 30, 60 and 120 minutes. The results are displayed in Table 1.

TABLE 1

| Compound | % ID/g liver | % ID/g kidney |
|---|---|---|
| 2 | 71 | 7 |
| 3 | 61 | 20 |
| 4 | 20 | 70 |

From the biodistribution results in table 1 it is apparent, that the presence of a PEG containing moiety that comprises 2 to 50 ethylene glycol units in compound 4—i.e. a compound according to claim 1, remarkably improves the biodistribution of the compound in comparison to compounds 2 and 3; the compound is preferably excreted via the renal system.

1. A compound comprising
i) a PEG containing moiety comprising 2 to 30 ethylene glycol units;
ii) an imageable moiety useful in PET imaging or SPECT imaging; and
a hydrophobic non-peptidic vector moiety not
having affinity for the Angiotensin II receptor for delivery of said compound to a disease associated target.

2. (canceled)

3. A compound according to claim 1 wherein said PEG containing moiety is a straight chain PEG containing moiety comprising one or more PEG units.

4. A compound according to claim 1 wherein said PEG containing moiety forms a linker between the imageable moiety and said non-peptidic vector moiety.

5. (canceled)

6. (canceled)

7. (canceled)

8. (canceled)

9. A compound according to claim 1 or 4 wherein said non-peptidic vector moiety is a small organic molecule of less than 1000 Da.

10. (canceled)

11. An imaging agent comprising the compound according to claim 1 or 4 and one or more pharmaceutically acceptable adjuvants, excipients or diluents.

12. (canceled)

13. A method of generating contrast enhanced images of a human or non-human animal body, comprising administering a compound according to claim 1.

14. A compound comprising
i) a PEG containing moiety comprising 2 to 30 ethylene glycol units;
ii) an imageable moiety useful in PET imaging or SPECT imaging; and
iii) a hydrophobic non-peptidic vector moiety for delivery of said compound to a disease associated target.

15. A compound comprising:
i) a PEG-containing moiety comprising 2 to 30 ethylene glycol units;
ii) an imageable moiety; and
iii) a hydrophobic non-peptidic vector moiety for delivery of said compound to a disease associated target.

16. A compound according to claim 1, 14, or 15, wherein said imageable moiety is ¹⁸F.

17. An imaging agent comprising the compound according to claim 14, 15 or 16 and one or more pharmaceutically acceptable adjuvants, excipients or diluents.

18. A method of generating contrast enhanced images of a human or non-human animal body, comprising administering a compound according to claim 17.

19. A compound according to claim 1, 14 or 15 wherein the PEG containing moiety comprises 2 to 6 ethylene glycol units.

20. A compound according to claim 16 wherein the PEG containing moiety comprises 2 to 6 ethylene glycol units.

21. A compound according to claim 1, 14 or 15 wherein the presence of PEG increases the amount of compound excreted via the renal system.

22. A compound according to claim 16 wherein the presence of PEG increases the amount of compound excreted via the renal system.

23. A compound according to claim 14 or 15 wherein the PEG containing moiety is a straight chain PEG containing moiety comprising one or more PEG units.

24. A compound according to claim 16 wherein said PEG containing moiety is a straight chain PEG containing moiety comprising one or more PEG units.

25. A compound according to claim 14 or 15 wherein the PEG containing moiety forms a linker between said imageable moiety and said non-peptidic vector moiety.

26. A compound according to claim 16 wherein the PEG containing moiety forms a linker between said imageable moiety and said non-peptidic vector moiety.
